(19) United States
(12) Patent Application Publication
(10) Pub. No.: US 2011/0015621 A1
(43) Pub. Date: Jan. 20, 2011

(54) LASER DEVICE FOR MINIMALLY INVASIVE TREATMENT OF SOFT TISSUE

(75) Inventor: Michael Kreindel, Richmond Hill (CA)

Correspondence Address: HARNESS, DICKEY & PIERCE, P.L.C., P.O. BOX 828, BLOOMFIELD HILLS, MI, 48303, US

(73) Assignee: INVASIX CORPORATION, Richmond Hill (CA)

(21) Appl. No.: 12/502,257

(22) Filed: Jul. 14, 2009

(51) Int. Cl. A61B 18/22

(52) U.S. Cl. 606/15; 606/14

(57) ABSTRACT

A method and device for thermal fat destruction and collagen contraction The method comprises delivering optical energy into the adipose tissue under the skin to create thermal damage of fat, with an alteration of the overlying soft tissue contour and heating of dermis to sub-necrotic temperature to cause skin tightening. The device comprises a treatment cannula and tip connected to the cannula and optimizing light distribution for effective energy use.

### FIELD OF THE INVENTION

The invention relates to methods and device soft tissue thermal treatment.

### BACKGROUND OF THE INVENTION

Liposuction remains the number one cosmetic surgery procedure in North America. Liposuction is performed by inserting fenestrated cannulas into fat and removing the fat under vacuum pressure through the fenestrated openings in the cannula. Fat may also be destroyed by inserted ultrasonic probes directly into the fat causing cavitation or by using a reciprocating probe inserted into the fat.

Recently laser assisted liposuction has gained broad popularity due to the following combined effects created by laser:

Fat destruction

Blood vessel coagulation

Thermal collagen contraction

These allow performing more gentle aspiration with less bleeding and post surgery skin tightening.

Laser liposuction is based on thermal fat destruction, blood coagulation and collagen contraction induced by laser radiation delivered to the subcutaneous tissue through the optical fiber imbedded into the treatment cannula. U.S. Pat. No. 6,206,873 describes a method of lipolysis using laser radiation delivered through an optical fiber inserted into the hollow needle. Where the needle pierces the skin of a patient and bringing tip of the optical fiber into a subcutaneous adipose layer of the patient.

U.S. Patent application Publication No. US 20040034341 describes a method where laser radiation used for fat treatment is absorbed in the adipose tissue more than in water based tissue.

U.S. Patent application Publication No. US 20090076489 describes the use of an accelerometer for tracking position of the laser hand piece during the laser-assisted liposuction.

U.S. Patent application Publication No. US 20080306476 describes a method of skin and subcutaneous tissue heating using laser radiation and molding it to the new shape.

U.S. Patent application Publication No. US 20080188835 describes a device for cellulite and adipose tissue treatment where a laser fiber is incorporated into the aspiration cannula.

U.S. Pat. No. 6,206,873 describes method fat removing from the body where liposuction cannula is assembled with laser fiber for cutting adipose tissue and an irrigating system for cooling the fiber.

All above mentioned devices and methods are based on adipose tissue treatment by laser energy delivered through an optical fiber. Typical fiber diameter used for laser-assisted liposuction is in the range of 400-1000 microns that at 30W output power providing power density from about 4 kW/cm² to 24 kW/cm. Such high power density creates very high temperature in vicinity of the fiber and carbonization of the tissue that prevents energy propagation. Also high energy density may cause accidental skin burn if the laser fiber gets too close to the dermis.

Adipose tissue in the near infrared range (700 nm-1500 nm) has a low scattering coefficient and radiation propagates along the fiber axis for a few millimeters before it is absorbed. Tumescent anesthesia used in liposuction is based on saline which is also has low scattering properties and favors the directional propagation of light. Because fiber during the procedure is displaced in the same direction the fat volume over the fiber canal is over treated while surrounding tissue is not affected.

### SUMMARY OF THE INVENTION

A device is provided for thermal fat destruction and collagen remodeling of a body. The device has a cannula with at least one optical channel For delivering optical energy in the form of light into the body. A light emitting tip is connected to the cannula. The light emitting tip has a light emitting area larger than the cross-sectional area of the optical channel. A light source is connected to the cannula for providing light with a power sufficient to coagulate tissue in the vicinity of the light emitting tip.

The light source may be a laser.

The light source may be connected to the cannula with optical fiber.

The light generated by the light source may be in the spectral range of from 400 nm to 20 nm.

Light emitting tip may have a light emitting area in the range of 2 mm² to 200 mm².

The light emitting tip may be configured to direct at least pan of the light in a preferred direction different from a longitudinal axis of the cannula.

The light emitting tip may be configured to diffuse the optical energy over the tip of the light emitting surface.

The cannula may include a lumen for vacuum suction of coagulated tissue.

The cannula may include a thermal sensor.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a device according to the present invention with a hand piece connected;

FIG. 2 shows a treatment cannula according to the present invention that is inserted into the subcutaneous tissue;

FIG. 3 shows a treatment cannula according to the present invention that directs light in a preferred direction; and,

FIG. 4 shows treatment cannula according to the present invention that has a diffusing tip for scattering light around the tip.

### DETAILED DESCRIPTION OF PREFERRED EMBODIMENTS

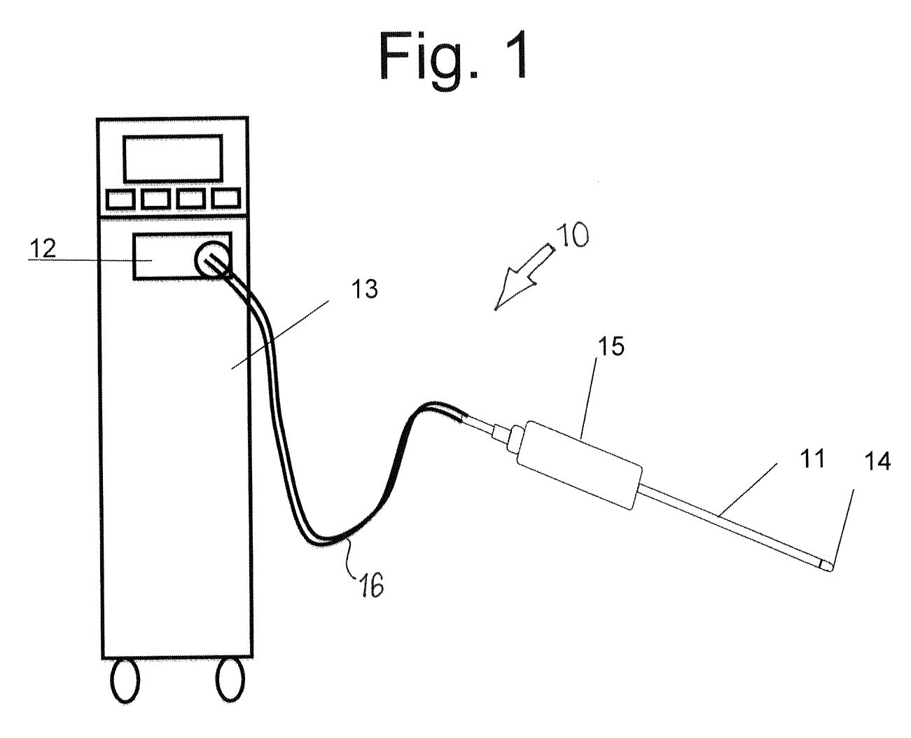

A device according to the present invention is generally indicated by reference 10 in FIG. 1. The device includes a light source 12 housed within a device console 13 and connected via an optical fiber 16 to a cannula 11. The cannula is configured to be inserted into a human body. The term “body” as used herein includes the face, neck and arms as well as other parts thereof. The cannula has a handle 15, and a light emitting tip 14 distal to the handle.

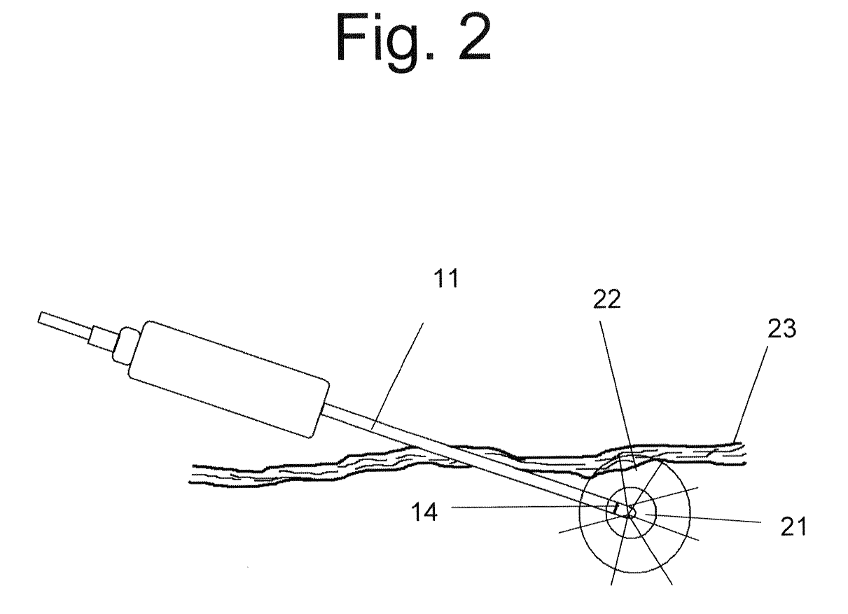

FIG. 2 depicts a light density gradient surrounding the tip 14. Immediately adjacent to tip 14 is a region 21 in which the light is intense enough to cause soft tissue coagulation. The density or intensity of the light decreases with distance from the tip 14 as shown by the region 22 where the intensity is consistent with necrotic tissue remodelling.

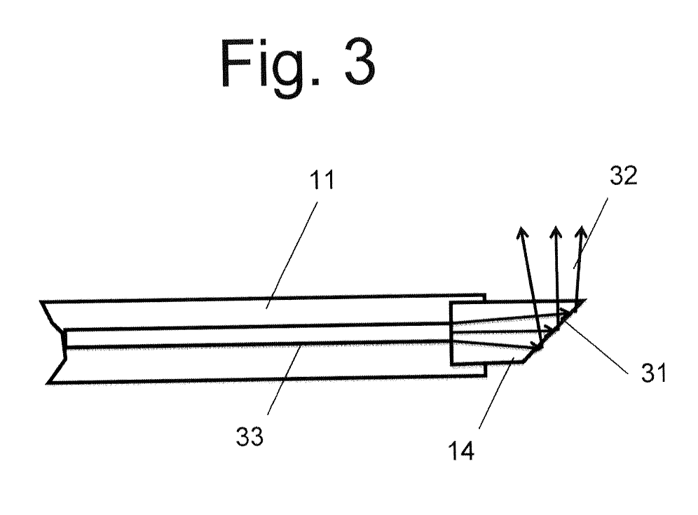

FIG. 3 illustrates in detail the light emitting tip 14, the end of the cannula 11 adjacent the light emitting tip 14 and an optical fiber 33 standing along the cannula 11. The optical fiber 33 would receive light from the optical fiber 16 associated with the device 10.

The tip 14 is configured to direct a light beam 32 emanating from the optical fiber 33 in a direction generally transverse to the axis of the cannula. Various arrangements may be used to cause the direction of the light beam 32. As illustrated in FIG. 3, the tip may have a reflecting area 31 of glass or crystal having an angle that reflects a significant part of the light in the desired direction. Alternatively, the tip may have a surface with a reflecting coating or utilize an external mirror attachable to the reflecting surface to re-direct the light beam 32. The reflecting surface can redirect light in more than one direction.

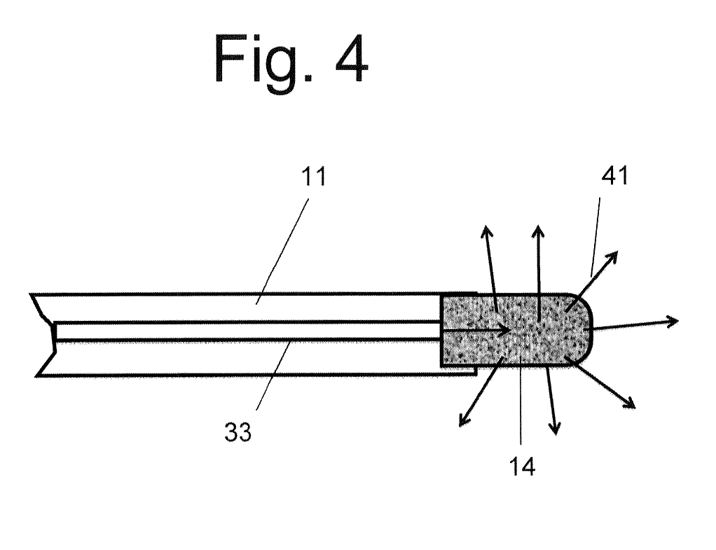

FIG. 4 shows another embodiment for the light emitting tip 14 extending from the cannula 11. The tip 14 is provided with an arrangement that scatters light in order to diffuse light 41 delivered to the tip 14 through the optical fiber 33 over the tip area. This may be accomplished for example by providing the tip 14 with a rough surface or with inclusions causing scattering of the light as it passes through the tip 14.

In using the system to treat subcutaneous adipose tissue and collect collagen the following exemplary parameter values may be selected:
a light spectrum having a wavelength of from 400 to 2000 nm;
an average output power of from about 1 W to about 200 W; and,
the delivered energy should create high enough temperature in the vicinity of the tip 14 of the cannula 11 to coagulate adipose tissue and/or contract collageneous tissue.

In general, the tip of the cannula has a light emitting are a larger than the cross-sectional area of the optical fiber 33 which delivers light to the tip 14.

The device may be used in a minimally invasive procedure where at least one optical fiber light guide is inserted into the soft tissue to he treated. The area of the light-emitting tip is configured to create an energy density sufficient to cause tissue coagulation but below that which would cause carbonization. The light energy density should be sufficient to create thermal damage to adipose tissue. The applied optical energy should be high enough to create adipocyte damage and/or collagen contraction in the region surrounding the tip 14 of the cannula. Destroying the tissue around the tip minimizes the amount of mechanical action required for the liposuction.

In the FIG. 3 embodiment, the tip has at least one reflective surface for changing the direction of the light delivered from the optical fiber 33. In this arrangement, the surface of the tip 14 which is directed toward any overlying skin should be transparent to transmit light toward the skin. The area of the transparent window should be large enough to yield a light power density below the level causing tissue carbonization. Preferably, the handle 15 will be ergonomically shaped so as to clearly indicate the direction of the light output.

Optical energy may be delivered in pulse mode to create fractional coagulation or ablation of the dermis to create dermis collagen remodeling and skin tightening. For fractional dermis treatment, the wavelengths used should preferably be absorbing in dermis but non-absorbing in fat.

In another embodiment, the light may have a different preferred direction than discussed above. For example, light may be directed into the body depth for treatment of deeper tissue such as facial. In the FIG. 4 embodiment, the size of the tip should preferably be in the range of 2 mm² to 200 mm². The diameter of the tip should preferably be in the range of 1 mm to 5 mm with a length in the range of from 1 mm to 20 mm.

The parameters of the light energy may be adjusted depending on the intended application. As mentioned above, light energy can be delivered as a sequence of pulses or in continuous mode. Although the spectral range of optical energy is in the range of 400 nm to 2,000 mm, the preferable range of wavelengths is in the near infrared range of 700 nm to 1600 nm. The optical power should be sufficient to heat tissue volumes from a few cubic centimeters for facial treatments up to a few liters for body fat treatment. Accordingly, optical power should be in the range of 1-30 W for treatment of delicate areas such as face, neck and knees. Optical power should be in the range of 30-200 W for abdominal area and other areas with larger volume.

The method of the invention may be used for example to achieve a reduction in body weight, local fat reduction, lipolysis, body reshaping, cellulite reduction, loose skin reduction, wrinkle treatment, body surface tightening, skin tightening and collagen remodeling.

The treated body zone may be protruded using vacuum suction or mechanically to localize the region being treated thereby reducing the risk of mechanical and thermal damage to deeper tissue structures.

The temperature required for collagen remodeling depends on heating time. For short millisecond range pulses the required temperature is 60-70° C. If treatment time is a few minutes, then the temperature should be in the range of 40-45° C. as required to cause collage remodeling without skin damage. Accordingly, the cannula may be provided with temperature sensors for measuring the temperature of the treated tissue to provide the required thermal effect and prevent it from overheating.

The cannula 11 may be connected through the optical fiber or light guide to a laser, a light emitting diode, a gas discharge lamp or an incandescent tamp. Lamp radiation may be passed through an optical filter to optimize the light spectrum. The light source may be located in the separate console or in the handle of the handpiece. Preferably, the light source is a diode laser but it may be other types of laser or light sources.

Light may be emitted in continuous wave, burst or pulse manner. Optical energy may be adjusted according to thermal sensor measurements.

As mentioned, the cannula 11 may have a temperature sensor for measuring tissue temperature in the vicinity of the tip 14 of the cannula. The signal from the temperature sensor may be used to adjust optical energy according to the measured temperature. For example, power may be switched off when a target tissue temperature is reached and switched on when the cannula is moved to a new locations with a lower temperature.

The cannula may have a lumen and vent hole in the vicinity of the treatment tip 14 for aspiration of coagulated tissue using a vacuum pump connected to the cannula 11.

The above description is intended in an illustrative rather than a restrictive sense. Variations to the described structures and methods may be apparent to persons of relevant skill in the art without departing from the spirit and scope of the invention as defined by the claims set out below.

1. A device for thermal fat destruction and collagen remodeling of a body comprising:
a cannula having at least one optical channel extending therealong to deliver optical energy in the form of light into the body;
a light emitting tip connected to the cannula and having a light emitting area larger than the cross-sectional area of the optical channel;
a light source connected to the cannula for providing light with a power sufficient to coagulate tissue in the vicinity of the light emitting tip.

2. A device according to claim 1 wherein the light source is a laser.

3. A device according to claim 1 wherein the light source is connected to the cannula with optical fiber.

4. A device according to claim 1 wherein the light generated by the light source is in the spectral range of 400 nm to 2000 nm.

5. A device according to claim 1 wherein the light emitting tip has a light emitting area in the range of from 2 mm² to 200 mm².

6. A device for thermal fat destruction and collagen contraction of a body comprising:
a treatment cannula having at least one optical channel for delivering energy in the form of light into the body;
a light emitting tip connected to the cannula for directing at least part of the light in a preferred direction which is different from a longitudinal axis of the cannula; and,
a light source connected to the cannula for providing light with a power that is high enough to coagulate tissue in the vicinity of light emitting tip.

7. A device according to claim 6 wherein the light source is a laser.

8. A device according to claim 6 wherein the tip has at least one reflecting element for re-directing the light in the preferred direction.

9. A device according to claim 6 wherein the light source is connected to the cannula with optical fiber.

10. A device according to claim 6 wherein the light generated by a light source is in the spectral range of from 400 nm to 2000 nm.

11. A device according to claim 6 wherein the light is delivered in a pulsed manner.

12. A device according to claim 6 wherein the optical energy is such as to create fractional thermal damage of deep dermis.

13. A device for thermal fat destruction and collagen contraction comprising:
a treatment cannula having at least one optical channel for delivering required optical energy into the body;
a light emitting tip connected to the cannula which diffuses the optical energy over the tip of the light emitting surface;
a light source connected to the cannula and providing light with power sufficient to coagulate tissue in the vicinity of the light emitting tip.

14. A device according to claim 13 wherein the light source is a laser.

15. A device according to claim 13 wherein the tip material has light diffusing inclusions.

16. A device according to claim 13 wherein the tip has a light diffusing surface.

17. A device according to claim 13 wherein the light source is connected to the cannula with optical fiber.

18. A device according to claim 13 wherein the light generated by the light source is in the spectral range of from 400 nm to 2000 nm.

19. A device according to any one of claims 1, 6 or 13 wherein the cannula comprises lumen for vacuum suction of coagulated tissue.

20. A device according to any one of claims 1, 6 or 13 wherein the cannula comprises thermal sensor.
